(19) United States
(12) Patent Application Publication
(10) Pub. No.: US 2013/0116572 A1
(43) Pub. Date: May 9, 2013

(54) METHOD AND APPARATUS FOR MEASURING 3-DIMENSIONAL OBJECT SHAPE BASED ON SHADOWS

(71) Applicant: Electronics and Telecommunications Research Institute, Daejeon (KR)

(72) Inventors: Seong-Ho SON, Daejeon (KR); Hyuk Je KIM, Daejeon (KR); Jong Moon LEE, Daejeon (KR); Bo Ra KIM, Daejeon (KR); Soon Ik JEON, Daejeon (KR); Hyung Do CHOI, Daejeon (KR)

(73) Assignee: Electronics and Telecommunications Research Institute, Daejeon (KR)

(21) Appl. No.: 13/645,654

(22) Filed: Oct. 5, 2012

(30) Foreign Application Priority Data
Nov. 8, 2011 (KR) 10-2011-0115888

(51) Int. Cl. A61B 6/00

(52) U.S. Cl. 600/473; 600/407; 600/476

(57) ABSTRACT

An apparatus measures a 3-Dimensional (3D) shape of an object. The apparatus includes a light emitting unit and a light receiving unit that are arranged to face each other with the object disposed in a space defined therebetween, wherein the light transmitting unit being arranged to scan the object, and the light receiving unit being arranged to sense a shadow of the scanned object formed thereon. The apparatus includes a rotation unit arranged to rotate the light emitting unit and the light receiving unit about a same rotational axis by a preset rotation angle until the rotation is fully made by a desired target angle and a shape restoration unit configured to measure a 3D shape of the object using shadows that are obtained for each preset rotation angle.

### RELATED APPLICATION(S)

This application claims the benefit of Korean Patent Application No. 10-2011-0115888, filed on Nov. 08, 2011, which is hereby incorporated by references as if fully set forth herein.

### FIELD OF THE INVENTION

The present invention relates to measurement of a 3-dimensional (3D) shape, and more particularly, to an apparatus and a method for measuring a 3D shape of an object based on shadow information on the object.

### BACKGROUND OF THE INVENTION

Generally, breast cancer is adenocarcinoma that occurs in breasts, and is a kind of cancer that occurs most frequently in woman. Much research is being conducted for diagnosing and treating breast cancer.

Presently, an early diagnosis method of breast cancer that is the most generally used is to detect a foreign tissue of breasts through a mechanical checkup. In addition, there is X-ray mammography which is a more detailed checkup method. However, the X-ray mammography may exert a bad influence on the human body due to frequent overexposure to X-ray. Another method of breast cancer is a microwave tomography that uses weak electromagnetic waves unharmful to the human body without using X-ray.

In the microwave tomography, weak electromagnetic waves are generated and pass through the inside of a checked-up body (i.e., breast), and thus, the internal image (indicative of distribution of permittivity and conductivity) of the checked-up body is predicted and restored, thereby diagnosing whether there is a tumor in the checked-up body. In such a microwave imaging apparatus, it is important to scan the surface of the checked-up body and measure an accurate 3D shape, for high-quality image restoration, and particularly, 3D image restoration. However, the checked-up body is generally required to be soaked in a water tank containing liquid and a micro imaging apparatus in which a radio wave transmission/reception antenna surrounds the checked-up body has a limitation in mechanical structure, and therefore it is complicated and difficult to measure a 3D shape using the existing scan system.

### SUMMARY OF THE INVENTION

In view of the above, the present invention provides an apparatus and a method, which measure the 3D shape of an object based on shadow information on the object.

In accordance with an aspect of the present invention, there is provided an apparatus for measuring a 3-Dimensional (3D) shape of an object, which includes: a light emitting unit and a light receiving unit that are arranged to face each other with the object disposed in a space defined therebetween, wherein the light transmitting unit being arranged to scan the object, and the light receiving unit being arranged to sense a shadow of the scanned object formed thereon; a rotation unit arranged to rotate the light emitting unit and the light receiving unit about a same rotational axis by a preset rotation angle until the rotation is fully made by a desired target angle; and a shape restoration unit configured to measure a 3D shape of the object using shadows that are obtained for each preset rotation angle.

In the apparatus, the light emitting unit includes an array of light transmitting elements in rows to irradiate light and scan the object.

In the apparatus, the light receiving unit includes an array of light sensing elements arranged in rows to sense the shadows of the scanned object by the rows of the light emitting elements.

In the apparatus, the 3D shape of the object is measured by bright and darkness information of the respective shadows.

The apparatus further includes an initialization unit configured to initialize the rotated positions of the light emitting unit and the light receiving unit to their starting positions when the object that is disposed in the space start to rotate.

In the apparatus, the shape restoration unit intersects bright and darkness portions of the shadows to obtain intersection portions of the shadows, each intersection portion being corresponded to a 3D sectional-surface shape of the object, thereby restoring the 3D shape of the object.

In the apparatus, the light emitting unit and the light receiving unit are deactivated when the rotation is fully made by the desired target angle.

In the apparatus, the light is one of visible light, infrared light, ultraviolet rays, and laser beam.

In accordance with another aspect of the present invention, there is provided a method for measuring a 3-Dimensional (3D) object shape, which includes: disposing an object on a space on a panel; scanning the object using light while rotating the panel by a preset angle once; collecting shadows of the scanned object; and restoring a 3D shape of the object using the collected shadows.

In the method, restoring a 3D shape of the object includes intersecting bright and darkness portions of the shadows to obtain intersection portions of the shadows, each intersection portion being corresponded to a 3D sectional-surface shape of the object, thereby restoring the 3D shape of the object.

In the method, the scanning of the object is continued until the panel is fully rotated by a desired target angle.

The method further includes initializing the rotated positions of the panel to its starting position when the object that is disposed in the space starts to rotate.

### BRIEF DESCRIPTION OF THE DRAWINGS

The above and other objects and features of the present invention will become apparent from the following description of embodiments given in conjunction with the accompanying drawings, in which:

FIG. 1 illustrates a block diagram of an apparatus for measuring a 3D shape of an object in accordance with an embodiment of the present invention;

FIG. 2 is a partially exploded perspective view of the light emitting unit and the light receiving unit in detail shown in FIG. 1;

FIG. 3 is a flowchart illustrating a method for measuring a 3D shape of an object in accordance with an embodiment of the present invention; and

FIGS. 4A to 4D are diagrams for describing a way of restoring a 3D shape of an object using bright and darkness information of a shadow in accordance with an embodiment of the present invention.

### DETAILED DESCRIPTION OF THE EMBODIMENTS

Hereinafter, an apparatus and method for obtaining 3D shape information of an object by using only shadow information of the object with reference to the accompanying drawings.

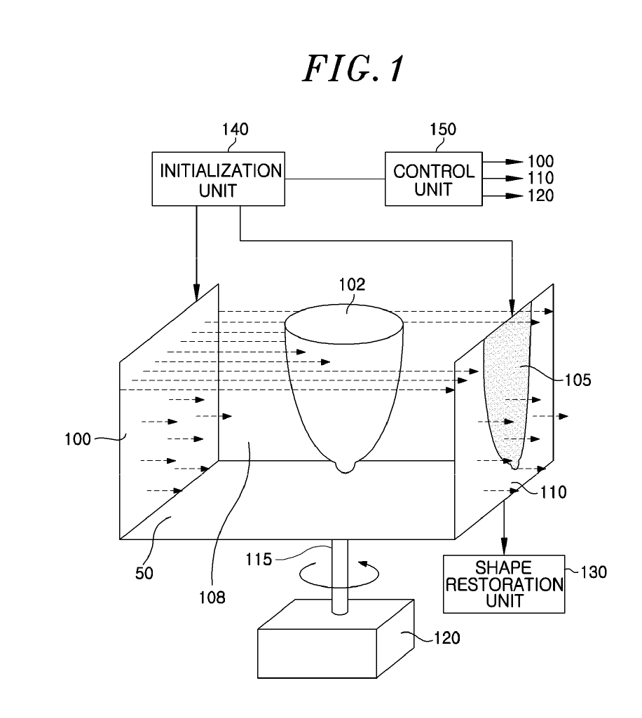

FIG. 1 illustrates a block diagram of an apparatus for measuring a 3D shape of an object in accordance with an embodiment of the present invention. Referring to FIG. 1, the apparatus for measuring a 3D shape of an object in accordance with an embodiment includes a light emitting unit 100, a light receiving unit 110, a panel 50, and a rotary actuator 120.

The light emitting unit 100 and the light receiving unit 110 are arranged to face each other with an object 102, e.g., a breast of a human body disposed in a space 108 defined therebetween. The panel 50 is arranged to fixedly support the light emitting unit 100 and the light receiving unit 110 at both sides thereof. The rotary actuator 120 has a rotation axis 115 that is connected to a bottom of the panel 50 to rotate the panel 50 by a preset rotation angle once until the panel 50 is fully rotated by a desired target angle, for example, 180 degrees.

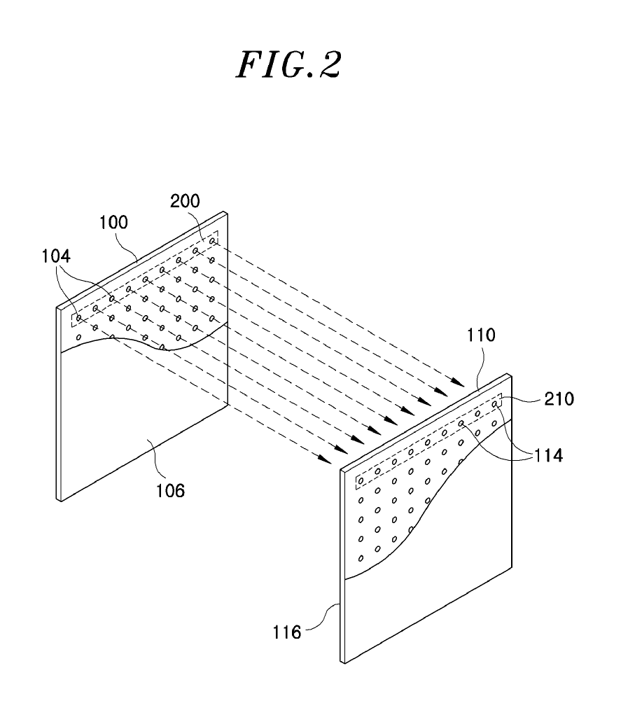

FIG. 2 illustrates partially exploded perspective views of the light emitting unit 100 and the light receiving unit 110 in detail.

As shown in FIG. 2, the light emitting unit 100 includes an array of light emitting elements 104 arranged in rows 200 to irradiate light and scan the object 102. The light irradiated from each row 200 of the light emitting elements 104 transmits a transparency surface 106 of the light emitting unit 100, which is configured to allow transmission of the light, and scan the surface of the object 102. In the embodiment, for example, the light emitted from the light emitting elements 104 may be visible light, infrared light, ultraviolet rays, laser beams, or the like.

In a similar manner, the light receiving unit 110 includes an array of light sensing elements 114 arranged in rows 210 to sense the light from the rows 200 of the light emitting elements 104. When the light emitting unit 100 are triggered to scan the object 102, the light passes by the object 102 while a portion of the light is blocked by the object 102 and is projected onto a transparency surface 116 of the light receiving unit 110, thereby casting a shadow 105 for the object 102 across the lighting receiving unit 110. The light sensing elements 114 then senses the bright and darkness of the shadow 105 across the lighting receiving unit 110 for respective rows 210.

Alternatively, the light emitting unit 100 may be substituted with a unit for generating a microwave, and the light receiving unit 110 may be substituted with a unit for detecting the frequency of the microwave from the microwave generating unit.

The apparatus further includes a shape restoration unit 130, an initialization unit 140 and a control unit 150.

The shape restoration unit 130 restores the 3D shape of the object 102 on the basis of the shadow information that is obtained by the array of the light sensing elements 114 for each rotation angles by the panel 50.

The initialization unit 140 initializes the rotated position of the panel 50 to its rotation starting position or its original position where the object 102 is firstly disposed in the space 108 for generating the 3D shape of the object 102 or the rotary actuator 140 starts to rotate the panel 50 under the control of the control unit 150.

The control unit 150 may activate sequentially or collectively the light emitting elements unit 104 to scan the object 102 by irradiating light from the rows of the light emitting elements 104. The light receiving unit 120 may also be controlled by the control unit 150 to activate the light sensing elements 114 in synchronization with the light emitting elements 104 in rows. Alternately, the light sensing elements 114 in the light receiving unit 110 may always remain activated.

The control unit 150 also deactivates the light emitting unit 100 and the light receiving unit 110 when the panel 50 has fully rotated by the desired target angle according to the driving of the rotary activator 120.

An operation of the apparatus for measuring a 3D shape having the above-described structure will now be described with reference to FIG. 2.

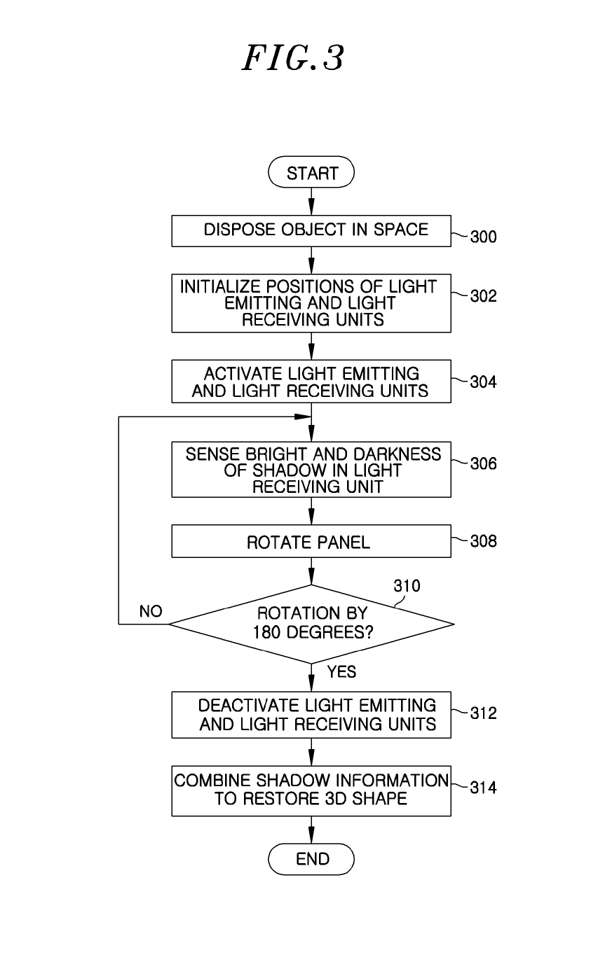

FIG. 3 is a flowchart illustrating a method for generating a 3D shape of the object in accordance with the embodiment of the present invention.

First, in operation 300, the object 102 is disposed at a central position of the space 108 on the panel 50 defined between the light emitting unit 100 and the light receiving unit 110.

In operation 302, the initialization unit 140 then initializes the positions of the light emitting unit 100 and the light receiving unit 110 by returning the panel 50 to its original position, if necessary.

Subsequently, in operation 304, the control unit 150 activates the light emitting unit 100 and the light receiving unit 110 so that each row of the light emitting elements 102 arranged in the light emitting unit 100 scans the object 102 by irradiating the light, whereby the shadow 105 of the object 102 is formed across the surface 114 of the light receiving unit 110.

In operation 306, the light sensing elements 114 senses bright and darkness of the shadow 105 for each row to produce shadow information having “0” and “1” indicative of the bright and darkness of the shadow 105. The shadow information is then provided to the shape restoration unit 130 for temporally storing thereof.

The control unit 150 controls the rotary actuator 120 to rotate the panel 50 by a preset rotation angle in operation 308. Therefore, the light emitting unit 100 and the light receiving unit 110 is rotated by the preset rotation angle.

The rotation is performed until the panel 50 is fully rotated by the desired target angle of 180 degrees in operation 310. When it is determined in operation 310 that the panel 50 has not rotated by 180 degrees, the method returns to operation 306 to repeatedly perform the rotation of the panel 50 until the panel 50 is fully rotated by 180 degrees. Therefore, the shape restoration unit 130 obtains the shadow information having bright and darkness portions of the shadow 105 for each rotation of the preset rotation angle, and temporally stores the shadow information.

When it is determined in operation 310 that the panel 50 has fully rotated by 180 degrees, the control unit 150 controls the rotary actuator 120 to stop the rotation of the rotation axis 115 and simultaneously deactivates the light emitting unit 100 and the light receiving unit 110 in operation 312.

Subsequently, the shape restoration unit 140 restores a 3D shape of the object 102 by combining the stored shadow information in operation 314.

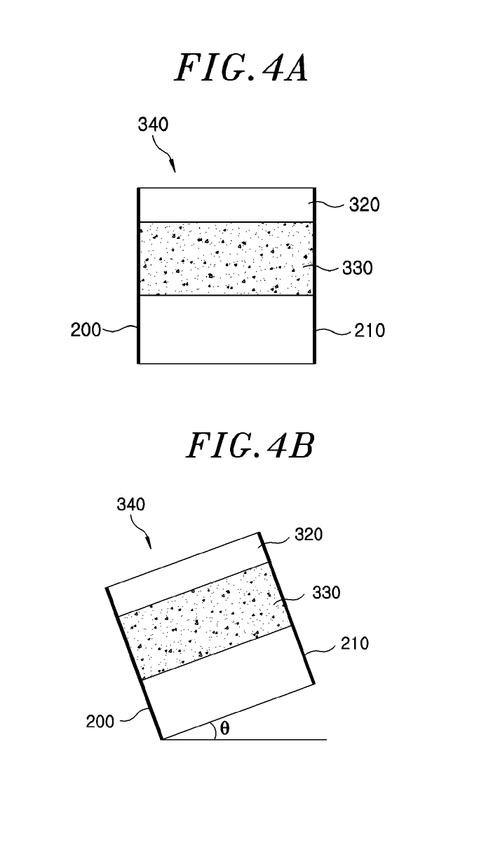

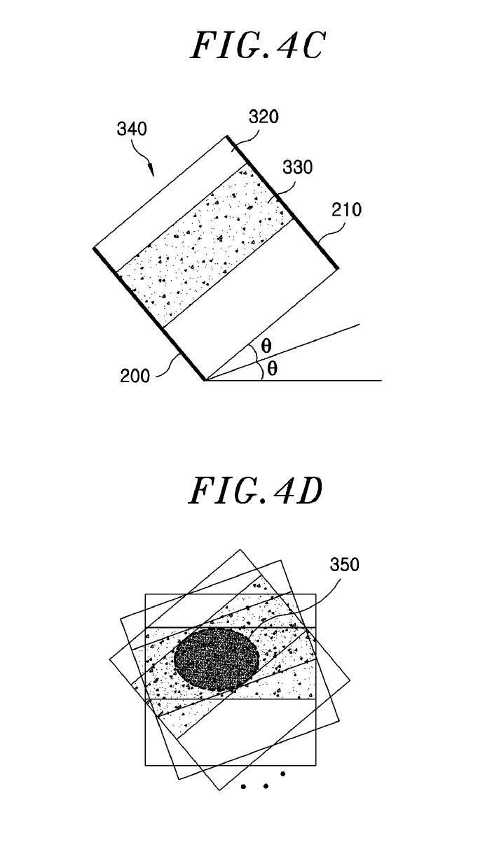

A process of a 3D shape restoration by the shape restoration unit 140 will now be described with reference to FIGS. 4A to 4D.

FIGS. 4A to 4D are diagrams illustrating a process of the 3D shape restoration of the object 102 in accordance with an embodiment of the present invention.

FIGS. 4A to 4C represent shadow information that has been obtained by scanning the object 102 using one row of the light emitting elements 104 at respective rotation angles θ. In FIGS. 4A to 4C, a virtual plane 340 is generated on a path on which the light from one row 200 of the light transmitting elements 104 is radiated onto a corresponding row 210 of the light sensing elements 114. The virtual plane 340 has a dark portion 330 in which a shadow is generated and a bright portion 320 except the shadow, both of which represent the shadow information of the object 102.

The obtained shadow information is then combined by intersecting the virtual planes 340 in FIGS. 4A to 4C, thereby obtaining an intersection portion 350 of the dark portions 330 as shown in FIG. 4D. The intersection portion 350 becomes the shape of a sectional surface of the object 102 to be restored. In this way, respective sectional-surface shapes of the object 102 are obtained for all rows of the light emitting and receiving elements 104 and 114. All the sectional-surface shapes are then combined and therefore a 3D shape of the object 102 is restored.

As described above, the embodiment measures the 3D shape of the object through a simple mechanical and driving structure without the interference of a radio wave transmission/reception antenna and a water tank containing liquid.

While the invention has been shown and described with respect to the embodiments, the present invention is not limited thereto. It will be understood by those skilled in the art that various changes and modifications may be made without departing from the scope of the invention as defined in the following claims.

What is claimed is:

1. An apparatus for measuring a 3-Dimensional (3D) shape of an object, including:
a light emitting unit and a light receiving unit that are arranged to face each other with the object disposed in a space defined therebetween, wherein the light transmitting unit being arranged to scan the object, and the light receiving unit being arranged to sense a shadow of the scanned object formed thereon;
a rotation unit arranged to rotate the light emitting unit and the light receiving unit about a same rotational axis by a preset rotation angle until the rotation is fully made by a desired target angle; and
a shape restoration unit configured to measure a 3D shape of the object using shadows that are obtained for each preset rotation angle.

2. The apparatus of claim 1, wherein the light emitting unit comprises an array of light transmitting elements in rows to irradiate light and scan the object.

3. The apparatus of claim 1, wherein the light receiving unit comprises an array of light sensing elements arranged in rows to sense the shadows of the scanned object by the rows of the light emitting elements.

4. The apparatus of claim 3, wherein the 3D shape of the object is measured by bright and darkness information of the respective shadows.

5. The apparatus of claim 1, further comprising an initialization unit configured to initialize the rotated positions of the light emitting unit and the light receiving unit to their starting positions when the object that is disposed in the space start to rotate.

6. The apparatus of claim 1, wherein the shape restoration unit intersects bright and darkness portions of the shadows to obtain intersection portions of the shadows, each intersection portion being corresponded to a 3D sectional-surface shape of the object, thereby restoring the 3D shape of the object.

7. The apparatus of claim 1, the light emitting unit and the light receiving unit are deactivated when the rotation is fully made by the desired target angle.

8. The apparatus of claim 2, wherein the light is one of visible light, infrared light, ultraviolet rays, and laser beam.

9. A method for measuring a 3-Dimensional (3D) object shape, the method comprising:
disposing an object on a space on a panel;
scanning the object using light while rotating the panel by a preset angle once;
collecting shadows of the scanned object; and
restoring a 3D shape of the object using the collected shadows.

10. The method of claim 9, wherein said restoring a 3D shape of the object comprises intersecting bright and darkness portions of the shadows to obtain intersection portions of the shadows, each intersection portion being corresponded to a 3D sectional-surface shape of the object, thereby restoring the 3D shape of the object.

11. The method of claim 9, wherein the scanning of the object is continued until the panel is fully rotated by a desired target angle.

12. The method of claim 11, further comprising initializing the rotated positions of the panel to its starting position when the object that is disposed in the space starts to rotate.
